(19) United States
(12) Patent Application Publication
(10) Pub. No.: US 2024/0050564 A1
(43) Pub. Date: Feb. 15, 2024

(54) COMBINATION THERAPY USING AN ANTI-FUCOSYL-GM1 ANTIBODY

(71) Applicant: BRISTOL-MYERS SQUIBB COMPANY, Princeton, NJ (US)

(72) Inventors: Yu LIU, Boston, MA (US); Sarah TANNENBAUM-DVIR, Princeton, NJ (US)

(21) Appl. No.: 18/271,579

(22) PCT Filed: Jan. 7, 2022
(86) PCT No.: PCT/US2022/011564

§ 371 Date: Jul. 10, 2023

Related U.S. Application Data
(60) Provisional application No. 63/135,479, filed on Jan. 8, 2021.

(51) Int. Cl. A61K 39/395; A61K 31/7048; A61K 31/282; A61P 35/00

(52) U.S. Cl. CPC A61K 39/3955; A61K 31/7048; A61K 31/282; A61P 35/00

(57) ABSTRACT

This disclosure provides combination therapy for treating a subject, such as a subject afflicted with lung cancer, such as small cell lung cancer, comprising administering to the subject various combinations of an anti-fucosyl-GM1 antibody, an immunomodulatory agent, such as a PD-1/PD-L1 antagonist, such as an antagonist anti-PD-1 or anti-PD-L1 antibody, carboplatin and etoposide.

### CROSS-REFERENCE TO RELATED APPLICATIONS

This application claims the benefit under 35 U.S.C. § 119(e) of U.S. Provisional Application Ser. No. 63/135,479 filed Jan. 8, 2021; the disclosure of which is incorporated herein by reference.

### SEQUENCE LISTING

The Sequence Listing filed electronically herewith is also hereby incorporated by reference in its entirety (File Name: 20220104_SEQL_13935WOPCT_GB; Date Created: 4 Jan. 2022; File Size: 20 KB).

### FIELD OF THE INVENTION

The present invention relates to improved methods of treatment of small cell lung cancer comprising administration of a combination of an antibody to fucosyl-GM1, an antibody to PD-1 or PD-L1, and a chemotherapeutic regimen.

### BACKGROUND OF THE INVENTION

Fucosyl-GM1 is a sphingolipid monosialoganglioside composed of a ceramide lipid component, which anchors the molecule in the cell membrane, and a carbohydrate component that is exposed at the cell surface. Carbohydrate antigens are the most abundantly expressed antigens on the cell surface of cancers (Feizi T. (1985) Nature 314:53-7). In some tumor types, such as small cell lung cancer (SCLC), initial responses to chemotherapy are impressive, but relapse rapidly follows. Intervention with novel immunotherapeutics may succeed in overcoming drug resistant relapse (Johnson D H. (1995) Lung Cancer 12 Suppl 3:S71-5). Several carbohydrate antigens, such as gangliosides GD3 and GD2, have been shown to function as effective targets for passive immunotherapy with mAbs (Irie R F and Morton D L (1986) PNAS 83:8694-8698; Houghton A N et al. (1985) PNAS 82:1242-1246). Ganglioside antigens have also been demonstrated to be effective targets for active immunotherapy with vaccines in clinical trials (Krug L M et al. (2004) Clinical Cancer Research 10:6094-6100; Dickler M N et al. (1999) Clinical Cancer Research 5:2773-2779; Livingston P O et al. (1994) J Clin. Oncol. 12:1036-44). Indeed, serum derived from SCLC patients who developed antibody titers to fucosyl-GM1 following vaccination with KLH conjugated antigen demonstrated specific binding to tumor cells and tumor specific complement dependent cytotoxicity (CDC). Anti-fucosyl-GM1 titer associated toxicities were mild and transient and three patients with limited-stage SCLC were relapse-free at 18, 24, and 30 months (Krug et al., supra; Dickler et al., supra).

Fucosyl-GM1 expression has been shown in a high percentage of SCLC cases and unlike other ganglioside antigens, fucosyl-GM1 has little or no expression in normal tissues (Nilsson et al. (1984) Glycoconjugate J 1:43-9; Krug et al., supra; Brezicka et al. (1989) Cancer Res. 49:1300-5; Zhangyi et al. (1997) Int. J. Cancer 73:42-49; Brezicka et al. (2000) Lung Cancer 28:29-36; Fredman et al. (1986) Biochim. Biophys. Acta 875: 316-23; Brezicka et al. (1991)APMIS 99:797-802; Nilsson et al. (1986) Cancer Res. 46:1403-7). The presence of fucosyl-GM1 has been demonstrated in culture media from SCLC cell lines, in tumor extracts and serum of nude mouse xenografts and in the serum of SCLC patients with extensive-stage disease (Vangsted et al. (1991) Cancer Res. 51:2879-84; Vangsted et al. (1994) Cancer Detect. Prev. 18:221-9). Fucosyl-GM1 expression has also been observed in a significant fraction of non-small cell lung cancer (NSCLC) samples. WO 07/067992. These reports provide convincing evidence for fucosyl-GM1 as a highly specific tumor antigen, which may be targeted by an immunotherapeutic.

An antibody that recognizes fucosyl-GM1 on cancer cells and directs their destruction, anti-fucosyl-GM1 mAb BMS-986012, has entered clinical trials for the treatment of subjects with relapsed/refractory small cell lung cancer (NCT02247349). See Molckovsky & Siu (2008) J Hematol. Oncol. 1:20. BMS-986012 is a non-fucosylated antibody and thus exhibits enhanced ADCC compared to an antibody with typical mammalian glycosylation. Although effective as a single agent, there exists a need for even more effective lung cancer therapy.

### SUMMARY OF THE INVENTION

The present invention provides combination therapy for treatment of lung cancer, most notably SCLC, involving initial rounds of induction therapy, e.g. four rounds, optionally followed by one or more rounds of maintenance therapy, wherein induction therapy comprises treatment with carboplatin, etoposide, anti-fucosyl-GM1 antibody and anti-PD-1/PD-L1 antibody, and maintenance therapy comprises treatment with anti-fucosyl-GM1 antibody and anti-PD-1/PD-L1 antibody.

In some embodiments, the anti-fucosyl-GM1 mAb competes with BMS-986012, comprises the same CDRs as BMS-986012, comprises the same heavy and light chain variable domains as BMS-986012, comprises the same heavy and light chains as BMS-986012, is BMS-986012, or is an antibody drug conjugate of BMS-986012. In one embodiment, the immunomodulatory agent is an anti-PD-1 mAb that competes with nivolumab (OPDIVO®), comprises the same CDRs as nivolumab, comprises the same heavy and light chain variable domains as nivolumab, or is nivolumab. In yet another embodiment, the immunomodulatory agent is an anti-PD-1 mAb that competes with pembrolizumab (KEYTRUDA®), comprises the same CDRs as pembrolizumab, comprises the same heavy and light chain variable domains as pembrolizumab, or is pembrolizumab. In a further embodiment, the immunomodulatory agent is an anti-PD-1 mAb that competes with cemiplimab-rwlc (LIBTAYO®), comprises the same CDRs as cemiplimab-rwlc, comprises the same heavy and light chain variable domains as cemiplimab-rwlc, or is cemiplimab-rwlc. In a still further embodiment, the immunomodulatory agent is an anti-PD-L1 mAb that competes with atezolizumab (TECENTRIQ®), comprises the same CDRs as atezolizumab, comprises the same heavy and light chain variable domains as atezolizumab, or is atezolizumab. In a yet further embodiment, the immunomodulatory agent is an anti-PD-L1 mAb that competes with durvalumab (IMFINZI®), comprises the same CDRs as durvalumab, comprises the same heavy and light chain variable domains as durvalumab, or is durvalumab. In an additional embodiment, the immunomodulatory agent is an anti-PD-L1 mAb that competes with avelumab (BAVENCIO®), comprises the same CDRs as avelumab, comprises the same heavy and light chain variable domains as avelumab, or is avelumab.

In some embodiments each round of induction therapy is 21 days long (Q3W). In some embodiments induction therapy comprises treatment with carboplatin at area under the curve (AUC) 5 mg/ml/min administered intravenously (iv) on day one of each cycle of induction therapy. In an alternative embodiment, cisplatin can be administered at 80 mg/m² in place of carboplatin. In some embodiments induction therapy comprises treatment with etoposide at 100 mg/m² iv on days one, two and three of each cycle of induction therapy. In some embodiments induction therapy comprises treatment with anti-fucosyl-GM1 mAb, e.g. BMS-986012, dosed at 420 mg iv on day one of each cycle of induction therapy. In some embodiments induction therapy comprises treatment with an anti-PD-1 mAb, e.g. nivolumab, dosed at 360 mg iv on day one of each cycle of induction therapy. In some embodiments all four therapeutic agents are administered during induction therapy as described in this paragraph.

In some embodiments each round of maintenance therapy is 28 days long (Q4W). In some embodiments maintenance therapy comprises treatment with anti-fucosyl-GM1 mAb, e.g. BMS-986012, dosed at 560 mg iv on day one of each cycle of maintenance therapy. In some embodiments maintenance therapy comprises treatment with an anti-PD-1 mAb, e.g. nivolumab, dosed at 480 mg iv on day one of each cycle of maintenance therapy. In some embodiments both therapeutic agents are administered during maintenance therapy as described in this paragraph.

In another aspect, the invention provides methods for treating a subject afflicted with small cell lung cancer (SCLC), e.g., a subject afflicted with extensive-stage SCLC (ES-SCLC), comprising administering to the subject a therapeutically effective combination of agents, such as monoclonal antibodies or antigen-binding portions thereof, that specifically bind to fucosyl-GM1 and an immunomodulatory target, such as PD-1 or PD-L1. In some embodiments, the anti-fucosyl-GM1 mAb is administered at 400 mg or 1000 mg Q3W or Q4W, the anti-PD-1 mAb is administered at 360 mg or 480 mg Q3W or Q4W, and the anti-PD-L1 mAb is administered at 1200 mg Q3W or Q4W.

In certain embodiments the anti-fucosyl-GM1 mAb, e.g. BMS-986012, is administered at 400 mg or 1000 mg and the anti-PD-1 mAb, e.g. nivolumab, is administered at 360 mg, both every three weeks (Q3W). In other embodiments the anti-fucosyl-GM1 mAb, e.g. BMS-986012, is administered at 400 mg or 1000 mg and the anti-PD-1 mAb, e.g. nivolumab, is administered at 480 mg, both every four weeks (Q4W). In further embodiments the anti-fucosyl-GM1 antibody and the anti-PD-1 antibody may be co-formulated in the same vial for combined administration.

In various embodiments, the method comprises one, two, three or four treatments, or is continued for as long as clinical benefit is observed or until unmanageable toxicity or disease progression occurs. In one embodiment, one or both of the antibodies is/are formulated for intravenous administration. The efficacy of the treatment methods provided herein can be assessed using any suitable means, such as reduction in size of the cancer, reduction in number of metastatic lesions over time, stable disease, partial response, and complete response.

In some embodiments the subject afflicted with SCLC has not previously been treated with a checkpoint inhibitor. In one embodiment, the subject has previously received an initial anti-cancer therapy. In another embodiment, the lung cancer is an advanced, metastatic, relapsed, and/or refractory lung cancer. In further embodiments the subject afflicted with SCLC has extensive-stage small cell lung cancer (ES-SCLC). In some embodiments the methods of the present invention are first-line treatment of lung cancers, such as SCLC. In other embodiments the methods of the present invention are second-line treatment of lung cancers, such as SCLC.

Other features and advantages of the instant invention will be apparent from the following detailed description and examples, which should not be construed as limiting. The contents of all cited references, including scientific articles, GenBank entries, patents and patent applications cited throughout this application are expressly incorporated herein by reference in their entireties.

### BRIEF DESCRIPTION OF THE DRAWINGS

FIG. 1 shows tumor growth in the mouse DMS79 tumor model as a function of treatment with anti-fucosyl-GM1 BMS-986012 and/or cisplatin. Median tumor volumes are presented for groups of 8 mice at each data point. See Example 1.

FIG. 2 shows tumor growth in the mouse DMS79 tumor model as a function of treatment with anti-fucosyl-GM1 BMS-986012 and/or etoposide. Median tumor volumes are presented for groups of 8 mice at each data point. See Example 2.

FIGS. 3A and 3B illustrate an exemplary combination therapy of the present invention. FIG. 3A provides dosing and administration details for a cycle of induction therapy and a cycle of maintenance therapy (Arm A), as described in greater detail at Example 5, as well as analogous cycles lacking treatment with anti-fucosyl-GM1 (Arm B). FIG. 3B provides a table graphically illustrating the dosing schedule for the cycles illustrated in FIG. 3A and outlined in Example 5.

### DETAILED DESCRIPTION OF THE INVENTION

BMS-986012 is a first-in-class fully human monoclonal antibody (mAb) that specifically binds to the fucosyl-GM1 ganglioside. BMS-986012 exhibits high-affinity and dose-dependent saturable binding to fucosyl-GM1 and shows no detectable antigen-specific binding to closely related molecule GM1. Because fucosyl-GM1 is preferentially found on the surface of lung cancer cells, BMS-986012 is particularly well suited to treating lung cancer, such as small cell lung cancer (SCLC).

BMS-986012 is non-fucosylated (lacking fucosylation on the Fc domain). The absence of the fucosyl group in BMS-986012 confers higher affinity for Fc receptors resulting in enhanced antibody-dependent cellular cytotoxicity (ADCC). Furthermore, the antibody was shown to mediate potent complement dependent cytotoxicity (CDC) as well as antibody-dependent cellular phagocytosis (ADCP). See, e.g., WO 2007/067992, the content of which is expressly incorporated herein by reference in its entirety.

Although BMS-986012 is effective as monotherapy in treatment of lung cancer, improved methods of treatment are always desired.

Programmed Cell Death 1 (PD-1) is a cell surface signaling receptor that plays a critical role in the regulation of T cell activation and tolerance. Keir et al. (2008) Ann. Rev. Immunol. 26:677-704. It is a type I transmembrane protein and together with BTLA, CTLA-4, ICOS and CD28, comprise the CD28 family of T cell co-stimulatory receptors. PD-1 is primarily expressed on activated T cells, B cells, and myeloid cells. Dong et al. (1999) Nat. Med. 5:1365-1369. It is also expressed on natural killer (NK) cells. Terme M et al. (2011) Cancer Res. 71:5393-5399. Binding of PD-1 by its ligands, PD-L1 and PD-L2, results in phosphorylation of the tyrosine residue in the proximal intracellular immune receptor tyrosine inhibitory domain, followed by recruitment of the phosphatase SHP-2, eventually resulting in down-regulation of T cell activation. One important role of PD-1 is to limit the activity of T cells in peripheral tissues at the time of an inflammatory response to infection, thus limiting the development of autoimmunity. Pardoll (2012) Nat. Rev. Cancer 12:252-264. Evidence of this negative regulatory role comes from the finding that PD-1 deficient mice develop lupus-like autoimmune diseases including arthritis and nephritis, along with cardiomyopathy. Nishimura et al. (1999) Immunity; 11:141-151; and Nishimura et al. (2001) Science 291:319-322. In the tumor setting, the consequence is the development of immune resistance within the tumor microenvironment. PD-1 is highly expressed on tumor infiltrating lymphocytes, and its ligands are up-regulated on the cell surface of many different tumors. Dong et al. (2002) Nat. Med. 8:793-800. Multiple murine cancer models have demonstrated that binding of ligand to PD-1 results in immune evasion. In addition, blockade of this interaction results in anti-tumor activity. Topalian et al. (2012) New Eng. J. Med. 366(26):2443-2454; Topalian et al. (2012) Curr. Opin. Immunol. 24:207-212; Brahmer et al. (2012) New Eng. J. Med. 366(26):2455-2465; Hamid et al. (2013) New Eng. J. Med. 369:134-144; Hamid & Carvajal (2013) Expert Opin. Biol. Ther. 13(6):847-861.

Without intending to be limited by theory, BMS-986012 may mediate killing of fucosyl-GM1-expressing lung cancer cells and thus reduce the concentration of shed fucosyl-GM1 in the tumor microenvironment. Early studies showed that gangliosides that are shed from tumor cells within the tumor microenvironment inhibit tumor-specific immune response. McKallip et al. (1999) J. Immunol. 163:3718. Such ganglioside-mediated suppression of anti-tumor immune response may be mediated, e.g., by a shift from IFN-γ production toward a more Th2-type T cell response (Crespo et al. (2006) J. Leukocyte. Biol. 79:586), and/or suppression of dendritic cell maturation (Wolff et al. (2002) Clin. Exp. Immunol. 130:441; Bennaceur et al. (2006) Int. Immunol. 18:879). Regardless of the detailed mechanism, reducing of the concentration of shed ganglioside by treatment with an anti-fucosyl-GM1 antibody would be expected to reduce this suppression of anti-tumor immune response, and consequently enhance the effectiveness of immunomodulatory agents, such as PD-1/PD-L1 antagonists, that act by enhancing this response.

Either or both of the above mentioned mechanisms of action may occur in the tumor microenvironment. Without intending to be limited by theory, combination therapy with an anti-fucosyl-GM1 antibody and a PD-1 antagonist may effectively treat tumors that would not otherwise be sufficiently immunogenic to generate an adequate anti-tumor immune response to support monotherapy with a PD-1/PD-L1 antagonist, and which would not be sufficiently eradicated by monotherapy with an anti-fucosyl-GM1 antibody either.

### I. Definitions

In order that the present disclosure may be more readily understood, certain terms are first defined. As used in this application, except as otherwise expressly provided herein, each of the following terms shall have the meaning set forth below. Additional definitions are set forth throughout the application.

“BMS-986012,” as used herein, refers to an anti-fucosyl-GM1 mAb comprising heavy chains of SEQ ID NO: 3 and light chains of SEQ ID NO: 4. BMS-986012 also comprises heavy chain variable domains of SEQ ID NO: 1 and light chain variable domains of SEQ ID NO: 2. BMS-986012 also comprises CDR sequences of SEQ ID NO: 5 (CDRH1), SEQ ID NO: 6 (CDRH2), SEQ ID NO: 7 (CDRH3), SEQ ID NO: 8 (CDRL1), SEQ ID NO: 9 (CDRL2), SEQ ID NO: 10 (CDRL3).

“Antagonist of PD-1/PD-L1” or equivalently “antagonist of PD-1” refers to any agent that blocks the interaction of PD-1 and PD-L1, such as antagonist antibodies specific to either PD-1 or PD-L1, antibody fragments thereof, soluble receptor constructs, nucleic acid-based inhibitors of the expression of the PD-1 and/or PD-L1 genes or mRNA translation, etc. Such agents include, but are not limited to, nivolumab, pembrolizumab, cemiplimab-rwlc, dostarlimab-gxly, zimberelimab, penpulimab, atezolizumab, durvalumab, avelumab and envafolimab. Nivolumab, also known as “BMS-936558,” refers to an anti-PD-1 mAb comprising heavy chains of SEQ ID NO: 13 and light chains of SEQ ID NO: 14. BMS-936558 also comprises heavy chain variable domains of SEQ ID NO: 11 and light chain variable domains of SEQ ID NO: 12. BMS-936558 also comprises CDR sequences of SEQ ID NO: 15 (CDRH1), SEQ ID NO: 16 (CDRH2), SEQ ID NO: 17 (CDRH3), SEQ ID NO: 18 (CDRL1), SEQ ID NO: 19 (CDRL2), SEQ ID NO: 20 (CDRL3). Sequences for pembrolizumab are provided in the sequences claimed in U.S. Pat. Nos. 8,354,509 and 8,900,587 (heavy chain CDRs 1, 2 and 3 are sequences 18, 19 and 20; light chain CDRs 1, 2 and 3 are sequences 15, 16 and 17, respectively; heavy chain sequence is residues 20-446 of sequence 31 and light chain sequence is residues 20-237 sequence 36), and also at CAS Registry No. 1374853-91-4, and at Pembrolizumab: Statement on a Nonproprietary Name Adopted by the USAN Council N13/140 (Nov. 27, 2013). Sequences for cemiplimab-rwlc are provided at WHO Drug Information Vol. 32, No. 2 (2018) Proposed INN: List 119 (CAS Registry No. 1801342-60-8). Sequences for dostarlimab-gxly are provided at WHO Drug Information Vol. 32, No. 2 (2018) Proposed INN: List 119 (CAS Registry No. 2022215-59-2). Sequences for zimberelimab are provided at WHO Drug Information Vol. 34, No. 2 (2020) Proposed INN: List 123 (CAS Registry No. 2259860-24-5). Sequences for penpulimab are provided at WHO Drug Information Vol. 34, No. 2 (2020) Proposed INN: List 123 (CAS Registry No. 2350298-92-7). Other anti-PD-1 antibodies under regulatory review may also find use in different embodiments of the present invention, including but not limited to, retifanlimab (WHO Drug Information Vol. 33, No. 2 (2019) Proposed INN: List 121 (CAS Registry No. 2079108-44-2), sintilimab (WHO Drug Information Vol. 32, No. 2 (2018) Proposed INN: List 119, CAS Registry No. 2072873-06-2), tislelizumab (WHO Drug Information Vol. 31, No. 2 (2017) Proposed INN: List 117, CAS Registry No. 1858168-59-8), toripalimab (WHO Drug Information Vol. 32, No. 2 (2018) Proposed INN: List 119, CAS Registry No. 1924598-82-2), geptanolimab (WHO Drug Information Vol. 34, No. 2 (2020) Proposed INN: List 123, CAS Registry No. 2348469-43-0), and serplulimab (WHO Drug Information Vol. 33, No. 2 (2019) Proposed INN: List 121, CAS Registry No. 2231029-82-4). Antibody sequences referred to herein are hereby incorporated by reference.

With regard to anti-PD-L1 antibodies, sequences for atezolizumab are provided at U.S. Pat. No. 8,217,149 (light chain variable domain sequence is sequence 21 and the heavy chain variable domain sequence is sequence 20 and or a variant thereof comprising an additional serine (S) residue between S117 and A118). Sequences for atezolizumab and durvalumab are provided at WHO Drug Information Vol. 29, No. 3 (2015) Recommended INN: List 74. Sequences for avelumab are provided at WHO Drug Information Vol. 30, No. 1 (2016) Recommended INN: List 75. Sequences for envafolimab are provided at WHO Drug Information Vol. 32, No. 4 (2018) Proposed INN: List 120 (CAS Registry No. 2102192-68-5). Other anti-PD-L1 antibodies under regulatory review may also find use in different embodiments of the present invention, including but not limited to, sugemalimab (WHO Drug Information Vol. 33, No. 4 (2019) Proposed INN: List 122, CAS Registry No. 2256084-03-2) and socazolimab (WHO Drug Information Vol. 35, No. 2 (2021) Proposed INN: List 122, CAS Registry No. 2305043-30-3), and even anti-PD-L1 antibodies not yet under regulatory review may be considered, such as cosibelimab (WHO Drug Information Vol. 33, No. 2 (2019) Proposed INN: List 121 (CAS Registry No. 2216751-26-5). Antibody sequences referred to herein are hereby incorporated by reference.

Target proteins referenced herein, such as PD-1, are intended to refer to their human orthologs of these proteins (e.g. huPD-1; NP 005009; GeneID 5133) unless otherwise indicated or clear from the context.

“Administering” refers to the physical introduction of a composition comprising a therapeutic agent to a subject, using any of the various methods and delivery systems known to those skilled in the art. Preferred routes of administration for the anti-fucosyl-GM1 antibody include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Alternative, non-parenteral routes include a topical, epidermal or mucosal route of administration, for example, intranasally, vaginally, rectally, sublingually or topically. Administering can also be performed, for example, once, a plurality of times, and/or over one or more extended periods. Dose intervals denominated in “days” are intended to represent approximately 24 hour intervals, but may vary slightly due to scheduling difficulties or other delays in administration.

“Concurrent” administration refers to dosing of two distinct agents, such as anti-fucosyl-GM1 and anti-PD-1 or anti-PD-L1 antibodies, at or around the same time rather than intentionally delaying administration of one of the agents. As such, concurrent administration includes simultaneous administration, e.g. when the agents are co-formulated or mixed prior to administration, and also includes administration of the two drugs within a convenient interval, typically during the same visit to a health care facility. Typically concurrent administration is performed on the same day, and excludes administration at separate visits to a health care facility on different days.

An “adverse event” (AE) as used herein is any unfavorable and generally unintended or undesirable sign (including an abnormal laboratory finding), symptom, or disease associated with the use of a medical treatment. A medical treatment may have one or more associated AEs and each AE may have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin that binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. Sequences of antibody heavy chains herein may comprise a C-terminal lysine (K) residue but that residue may be clipped off during manufacture, entirely or partially, or it may be removed from the genetic construct used to produce the antibody so as to avoid potential heterogeneity arising from the aforementioned clipping. Both heavy chain sequences provided herein (SEQ ID NOs: 3 and 13) do not include C-terminal lysine residues.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man.

An “isolated antibody” refers to an Ab that is substantially free of other Abs having different antigenic specificities (e.g., an isolated Ab that binds specifically to fucosyl-GM1 is substantially free of Abs that bind specifically to antigens other than fucosyl-GM1). Moreover, an isolated Ab may be substantially free of other cellular material and/or chemicals.

The term “monoclonal antibody” (“mAb”) refers to a non-naturally occurring preparation of Ab molecules of single molecular composition, i.e., Ab molecules whose primary sequences are essentially identical, and which exhibits a single binding specificity and affinity for a particular epitope. A mAb is an example of an isolated Ab. MAbs may be produced by hybridoma, recombinant, transgenic or other techniques known to those skilled in the art.

A “human” antibody (HuMAb) refers to an Ab having variable regions in which both the framework and CDR regions are derived from human germline immunoglobulin sequences. Furthermore, if the Ab contains a constant region, the constant region also is derived from human germline immunoglobulin sequences. The human Abs of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include Abs in which CDR sequences derived from the germline of another mammalian species, such as a mouse, have been grafted onto human framework sequences. The terms “human” Abs and “fully human” Abs and are used synonymously.

A “humanized antibody” refers to an Ab in which some, most or all of the amino acids outside the CDR domains of a non-human Ab are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the Ab to bind to a particular antigen. A “humanized” Ab retains an antigenic specificity similar to that of the original Ab.

A “checkpoint inhibitor” refers to a therapeutic agent used to treat cancer that acts by blocking the activity “checkpoint” proteins that are made by some types of immune system cells, such as T cells, and some cancer cells. These checkpoint proteins suppress immune responses, e.g. to prevent undesirable immune pathology, but their actions may also prevent immunosurveillance that might otherwise eradicate or reduce tumor formation. Checkpoint inhibitors include, but are not limited to, therapeutic antagonist antibodies to PD-1, PD-L1, CTLA-4, TIGIT, LAG3, TIM3, VISTA and BTLA. Qin et al. (2019) Mol. Cancer 18:155.

A “chimeric antibody” refers to an Ab in which the variable regions are derived from one species and the constant regions are derived from another species, such as an Ab in which the variable regions are derived from a mouse Ab and the constant regions are derived from a human Ab.

An “anti-antigen” Ab refers to an Ab that binds specifically to the antigen. For example, an anti-fucosyl-GM1 Ab binds specifically to fucosyl-GM1.

An “antigen-binding portion” of an Ab (also called an “antigen-binding fragment”) refers to one or more fragments of an Ab that retain the ability to bind specifically to the same antigen bound by the whole Ab.

A “cancer” refers a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. The terms, “cancer,” “tumor,” and “neoplasm,” are used interchangeably herein.

A “subject” includes any human or nonhuman animal. The term “nonhuman animal” includes, but is not limited to, vertebrates such as nonhuman primates, sheep, dogs, and rodents such as mice, rats and guinea pigs. In preferred embodiments, the subject is a human. The terms, “subject” and “patient” are used interchangeably herein.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” promotes cancer regression in a subject. In preferred embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount of an anti-cancer agent preferably inhibits cell growth or tumor growth by at least about 20%, more preferably by at least about 40%, even more preferably by at least about 60%, and still more preferably by at least about 80% relative to untreated subjects. In other preferred embodiments of the invention, tumor regression may be observed and continue for a period of at least about 20 days, more preferably at least about 40 days, or even more preferably at least about 60 days. Notwithstanding these ultimate measurements of therapeutic effectiveness, evaluation of immunotherapeutic drugs must also make allowance for “immune-related” response patterns.

A therapeutically effective amount of a drug includes a “prophylactically effective amount,” which is any amount of the drug that, when administered alone or in combination with an anti-neoplastic agent to a subject at risk of developing a cancer (e.g., a subject having a pre-malignant condition) or of suffering a recurrence of cancer, inhibits the development or recurrence of the cancer. In preferred embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “approximately” refer to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within 1 or more than 1 standard deviation per the practice in the art. Alternatively, “about” or “approximately” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “approximately” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated. Such ranges further include the values as the boundaries of the ranges.

Various aspects of the invention are described in further detail in the following subsections.

### II. Anti-Fucosyl-GM1 Antibodies

HuMAbs that bind specifically to fucosyl-GM1 with high affinity have been disclosed in U.S. Pat. No. 8,383,118 and WO 2007/067992 (e.g., human monoclonal antibodies 5B1, 5B1a, 7D4, 7E4, 13B8 and 18D5). Each of the HuMAbs disclosed in U.S. Pat. No. 8,383,118 has been demonstrated to exhibit one or more desirable functional properties: (1) specifically binds to fucosyl-GM1; (2) binds to fucosyl-GM1 with high affinity (for example with a K_{D} of 1×10⁻⁷ M or less); (c) binds to the human small cell lung cancer cell line DMS-79 (Human SCLC ATCC #CRL-2049); and (d) inhibit growth of tumor cells in vitro or in vivo. Preferably, the antibody binds to fucosyl-GM1 with a K_{D} of 5×10⁻⁸ M or less, binds to fucosyl-GM1 with a K_{D} of 1×10⁻⁸ M or less, binds to fucosyl-GM1 with a K_{D} of 5×10⁻⁹ M or less, or binds to fucosyl-GM1 with a K_{D} of between 1×10⁻⁸ M and 1×10⁻¹⁹ M or less. Standard assays to evaluate the binding ability of the antibodies toward fucosyl-GM1 are known in the art, including for example, ELISAs, Western blots and RIAs. The binding kinetics (e.g., binding affinity) of the antibodies also can be assessed by standard assays known in the art, such as by ELISA, Scatchard and Biacore analysis.

A preferred anti-fucosyl-GM1 Ab is BMS-986012 (also referred to as MDX-1110 or 7E4). Anti-fucosyl-GM1 Abs usable in the disclosed methods also include isolated Abs that bind specifically to fucosyl-GM1 and cross-compete for binding to fucosyl-GM1 with BMS-986012 (see, e.g., U.S. Pat. No. 8,383,118; WO 2007/067992). The ability of Abs to cross-compete for binding to an antigen indicates that these Abs bind to the same epitope region of the antigen and sterically hinder the binding of other cross-competing Abs to that particular epitope region. These cross-competing Abs are expected to have functional properties very similar those of BMS-986012 by virtue of their binding to the same epitope region of fucosyl-GM1. Cross-competing Abs can be readily identified based on their ability to cross-compete with BMS-986012 in standard fucosyl-GM1 binding assays such as Biacore analysis, ELISA assays or flow cytometry (see, e.g., WO 2013/173223).

For administration to human subjects, these Abs are preferably chimeric Abs, or more preferably humanized or human Abs. Such chimeric, humanized or human mAbs can be prepared and isolated by methods well known in the art. In some, but not all, embodiments anti-fucosyl-GM1 Abs usable in the methods of the disclosed invention also include antigen-binding portions of the above Abs. It has been amply demonstrated that the antigen-binding function of an Ab can be performed by fragments of a full-length Ab. Examples of binding fragments encompassed within the term “antigen-binding portion” of an Ab include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; and (iv) a Fv fragment consisting of the V_{L} and V_{H} domains of a single arm of an Ab. Anti-fucosyl-GM1 antibodies (or V_{H} and/or V_{L} domains derived therefrom) suitable for use in the invention can be generated using methods well known in the art. In other embodiments, e.g. in which effector function is important to activity, antibody fragments may not be suitable for use in the methods of the present invention.

An exemplary anti-fucosyl-GM1 antibody is BMS-986012 comprising heavy and light chains comprising the sequences shown in SEQ ID NOs: 3 and 4, respectively.

In other embodiments, the antibody has heavy and light chain CDRs or variable regions of BMS-986012. Accordingly, in one embodiment, the antibody comprises CDR1, CDR2, and CDR3 domains of the VH of BMS-986012 having the sequence set forth in SEQ ID NO: 1, and CDR1, CDR2 and CDR3 domains of the VL of BMS-986012 having the sequence set forth in SEQ ID NO: 2. In another embodiment, the antibody comprises the heavy chain CDR1, CDR2 and CDR3 domains comprising the sequences set forth in SEQ ID NOs: 5, 6, and 7, respectively, and the light chain CDR1, CDR2 and CDR3 domains comprising the sequences set forth in SEQ ID NOs: 8, 9, and 10, respectively. In another embodiment, the antibody comprises VH and VL regions comprising the amino acid sequences set forth in SEQ ID NO: 1 and SEQ ID NO: 2, respectively. In another embodiment, the antibody competes for binding with and/or binds to the same epitope on fucosyl-GM1 as the above-mentioned antibodies. In another embodiment, the antibody has at least about 90% variable region amino acid sequence identity with the above-mentioned antibodies (e.g., at least about 90%, 95% or 99% variable region identity with SEQ ID NO: 1 or SEQ ID NO: 2).

### III. PD-1 and PD-L1 Antagonists as Immunomodulatory Agents

Suitable PD-1 antagonists for use in the methods described herein, include, without limitation, ligands, antibodies (e.g., monoclonal antibodies and bispecific antibodies), and multivalent agents. In one embodiment, the PD-1 antagonist is a fusion protein, e.g., an Fc fusion protein, such as AMP-244. In one embodiment, the PD-1 antagonist is an anti-PD-1 or anti-PD-L1 antibody. See Twomey & Zhang (2021) The AAPS Journal 23:39.

An exemplary anti-PD-1 antibody is OPDIVO®/nivolumab (BMS-936558) or an antibody that comprises the CDRs or variable regions of one of antibodies 17D8, 2D3, 4H1, 5C4, 7D3, 5F4 and 4A11 described in WO 2006/121168. Sequences for nivolumab are provided at SEQ ID NOs: 11-21. In certain other embodiments, an anti-PD-1 antibody is MK-3475 (KEYTRUDA®/pembrolizumab/formerly lambrolizumab) described in WO 2012/145493 and claimed in U.S. Pat. Nos. 8,354,509 and 8,900,587. Cemiplimab-rwlc, whose sequence is found at WHO Drug Information Vol. 32, No. 2 (2018) Proposed INN: List 119 (CAS Registry No. 1801342-60-8), can also be used. An anti-PD-1 antibody that competes for binding with, and/or binds to the same epitope on PD-1 as one of these antibodies may also be used in combination treatments of the present invention.

Anti-PD-L1 antibodies may also be used in some embodiments of the present invention. Atezolizumab and durvalumab, whose sequences are found at WHO Drug Information Vol. 29, No. 3 (2015) Recommended INN: List 74. Avelumab, whose sequence is found at WHO Drug Information Vol. 30, No. 1 (2016) Recommended INN: List 75, can also be used. Anti-PD-L1 antibodies that compete with and/or bind to the same epitope as that of any of these antibodies may also be used in combination treatments of the present invention.

The ability of Abs to cross-compete for binding to an antigen indicates that these Abs bind to the same epitope region of the antigen and sterically hinder the binding of other cross-competing Abs to that particular epitope region. These cross-competing Abs are expected to have functional properties very similar those of the anti-PD-1 and anti-PD-L1 antibodies provided above by virtue of their binding to the same epitope region of PD-1 and PD-L1, respectively. Cross-competing Abs can be readily identified based on their ability to cross-compete with the anti-PD-1 and anti-PD-L1 antibodies provided above in standard binding assays such as Biacore analysis, ELISA assays or flow cytometry.

For administration to human subjects, these Abs are preferably chimeric Abs, or more preferably humanized or human Abs. Such chimeric, humanized or human mAbs can be prepared and isolated by methods well known in the art. In some, but not all, embodiments, anti-PD-1 and anti-PD-L1 Abs usable in the methods of the disclosed invention also include antigen-binding portions of the above Abs. Examples of binding fragments encompassed within the term “antigen-binding portion” of an Ab include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; and (iv) a FAT fragment consisting of the V_{L} and V_{H} domains of a single arm of an Ab.

### IV. Pharmaceutical Compositions

Therapeutic agents (e.g., anti-fucosyl-GM1 antibodies and/or anti-PD-1 or anti-PD-L1 antibodies, or antigen binding fragments thereof) of the present invention may be constituted in a composition, e.g., a pharmaceutical composition containing and a pharmaceutically acceptable carrier. Pharmaceutical compositions of the present invention include both individual antibodies and co-formulations.

As used herein, a “pharmaceutically acceptable carrier” includes any and all solvents, dispersion media, coatings, antibacterial and antifungal agents, isotonic and absorption delaying agents, and the like that are physiologically compatible. “Pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions (e.g., comprising an anti-fucosyl-GM1 antibody). Preferably, the carrier for a composition containing an Ab is suitable for intravenous, intramuscular, subcutaneous, parenteral, spinal or epidermal administration (e.g., by injection or infusion). A pharmaceutical composition of the invention may include one or more pharmaceutically acceptable salts, anti-oxidant, aqueous and non-aqueous carriers, and/or adjuvants such as preservatives, wetting agents, emulsifying agents and dispersing agents.

Liquid compositions for parenteral administration can be formulated for administration by injection or continuous infusion. Routes of administration by injection or infusion include intravenous, intraperitoneal, intramuscular, intrathecal and subcutaneous. In one embodiment, the anti-fucosyl-GM1 antibody is administered intravenously.

### V. Methods of Treatment

Provided herein are clinical methods for treating a lung cancer (e.g., small cell lung cancer) in a subject (e.g., a human subject), comprising administering to the subject a therapeutically effective amounts of an anti-fucosyl-GM1 antibody and an anti-PD1 antibody. In one embodiment, the subject has previously received an initial anti-cancer therapy. In another embodiment, the lung cancer is an advanced, metastatic, relapsed, and/or refractory lung cancer.

In another embodiment, the antibody is administered as a first line of treatment (e.g., the initial or first treatment). In another embodiment, the antibody is administered as a second line of treatment (e.g., after the initial or first treatment, including after relapse and/or where the first treatment has failed).

In certain specific embodiments, the antibody is administered according to at least one of the following dosing regimens: (a) about 400 mg of the antibody every 3 weeks; and (e) about 1000 mg of the antibody every 3 weeks. In one embodiment antibody is administered at a dose between 400 and 1000 mg, inclusive. Whether stated or not, any dose range recited herein is intended to be inclusive, i.e. it the doses recited as the boundaries of the ranges are included within the recited dosing range. Preferably, administration of the antibody induces a durable clinical response in the subject. Optionally, administration of the antibody is continued for as long as clinical benefit is observed or until unmanageable toxicity or disease progression occurs. The efficacy of the treatment methods provided herein can be assessed using any suitable means. In one embodiment, the treatment produces at least one therapeutic effect selected from the group consisting of reduction in size of the cancer, reduction in number of metastatic lesions over time, stable disease, partial response, and complete response.

Patients treated according to the methods disclosed herein preferably experience improvement in at least one sign of cancer. In one embodiment, improvement is measured by a reduction in the quantity and/or size of measurable tumor lesions. In another embodiment, lesions can be measured on chest x-rays or CT or MRI films. In another embodiment, cytology or histology can be used to evaluate responsiveness to a therapy.

In one embodiment, the patient treated exhibits a complete response (CR), a partial response (PR), or stable disease (SD). In another embodiment, the patient treated experiences tumor shrinkage and/or decrease in growth rate, i.e., suppression of tumor growth. In another embodiment, unwanted cell proliferation is reduced or inhibited. In yet another embodiment, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

### VI. Kits and Unit Dosage Forms

Also provided herein are kits that include a pharmaceutical composition containing an anti-fucosyl-GM1 antibody (such as BMS-986012) and an anti-PD-1 antibody (such as BMS-936558), and a pharmaceutically acceptable carrier, in a therapeutically effective amount adapted for use in the preceding methods.

The kits optionally can also include instructions, e.g., comprising administration schedules, to allow a practitioner (e.g., a physician or a nurse), or a patient, to administer the composition contained therein to a patient having a cancer (e.g., a lung cancer). The kit can also include a syringe.

Optionally, the kits include multiple packages of the single-dose pharmaceutical compositions each containing an effective amount of the antibody for a single administration in accordance with the methods provided above. Instruments or devices necessary for administering the pharmaceutical composition(s) also may be included in the kits. For instance, a kit may provide one or more pre-filled syringes containing an amount of the antibody.

The following examples are merely illustrative and should not be construed as limiting the scope of this disclosure in any way as many variations and equivalents will become apparent to those skilled in the art upon reading the present disclosure.

The contents of all references, GenBank entries, patents and published patent applications cited throughout this application are expressly incorporated herein by reference.

### Example 1

### Combination Therapy—Anti-Fucosyl-GM1 & Cisplatin

A combination therapy method of the present invention involving antibody to fucosyl-GM1 and cisplatin was tested in the DMS-79 SCLC tumor model in mice (N=9 mice per group). Bepler et al. (1989) Oncogene 4:45; Pettengill (1980) Cancer 45: 906; Pettengill et al. (1980) Exp. Cell Biol. 48:279. Briefly, DMS79 cells were cultured in RPMI with 10% fetal bovine serum (FBS), 2 mM L-glutamine, 15 mg/L sodium bicarbonate, 4.5 g/L glucose, 10 mM HEPES, and 10 μM NaPyr prior to subcutaneous implantation in the right flanks of male C.B17 SCID mice (5 million cells in 0.1 mL phosphate-buffered saline (PBS) and 0.1 mL MATRIGEL® gelatinous protein mixture per flank). When tumors reached a mean or median size of 80-155 mm³ estimated by L×W×H/2 using a digital calipers, mice were randomized into treatment groups (N=8 mice per group).

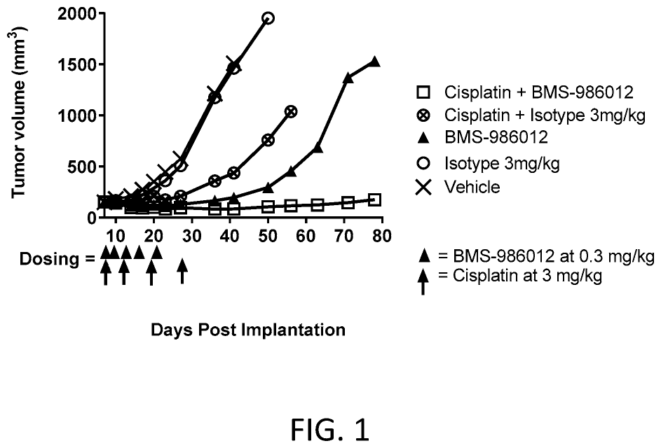

Anti-fucosyl-GM1 antibody BMS-986012 was administered at 0.3 mg/kg i.p. to mice on days 7, 10, 13, 17 and 21 post implantation, and cisplatin was administered at 3 mg/kg on days 7, 14, 21 and 28. Because fucosyl-GM1 is a ganglioside rather than a protein, and is the same in mice as in humans, there was no need to use a “mouse surrogate” for mouse studies. Cisplatin was administered in combination with BMS-986012 and with an isotype control antibody at 3 mg/kg as a control. Additional controls included the isotype control antibody alone and a vehicle control. Results are provided at FIG. 1. While both anti-fucosyl-GM1 antibody treatment and cisplatin treatment are effective as monotherapy to reduce tumor growth, the combination is significantly more effective—almost stopping tumor growth entirely.

### Example 2

### Combination Therapy—Anti-Fucosyl-GM1 & Etoposide

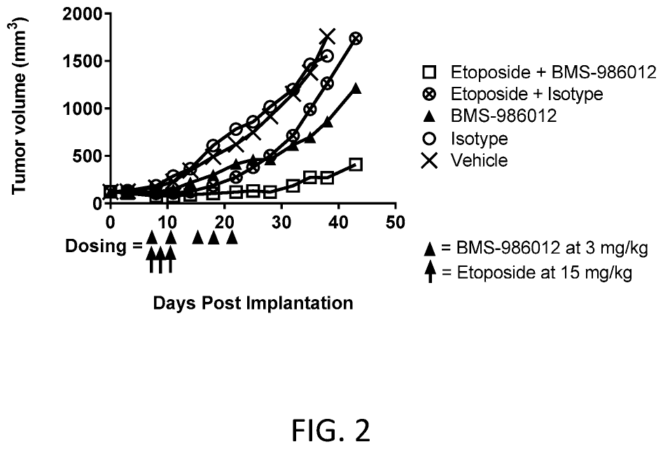

A combination therapy method of the present invention involving antibody to fucosyl-GM1 and etoposide was tested in the DMS-79 SCLC tumor model in mice (N=9 mice per group), essentially as described in Example 1. Anti-fucosyl-GM1 antibody BMS-986012 was administered at 3 mg/kg i.p. to mice on days 7, 11, 15, 18 and 21 post-implantation, and etoposide was administered at 15 mg/kg i.p. on days 7, 9 and 11. Etoposide was administered in combination with BMS-986012 and with an isotype control antibody at 3 mg/kg as a control. Additional controls included the isotype control antibody alone and a vehicle control. Results are provided at FIG. 2. While both anti-fucosyl-GM1 antibody treatment and etoposide treatment are somewhat effective as monotherapy to reduce tumor growth, the combination is more effective.

### Example 3

### Treatment of Human Subjects with Anti-Fucosyl-GM1 and Etoposide or Cisplatin

Human subjects were treated with a combination of anti-fucosyl-GM1 mAb BMS-986012 and cisplatin (or carboplatin) and etoposide in a Phase 1/2 Study (NCT02815592) to assess safety, essentially as follows. Previously untreated patients with extensive-stage small cell lung cancer (ES-SCLC) (n=14) were treated intravenously on day 1 with 400 mg (n=12) or 1000 mg (n=2) BMS-986012, combined with either cisplatin 80 mg/m² (part 1) or carboplatin area under the curve (AUC) 5 (part 2) on day 1, plus etoposide 100 mg/m² on days 1, 2, and 3 (both parts) over four 21-day cycles, followed by 400 mg or 1000 mg BMS-986012 monotherapy Q3W as maintenance.

Of the fourteen patients received BMS-986012 combined with platinum/etoposide, 11 continued into the monotherapy period. Three patients did not continue into the monotherapy period due to disease progression (n=2) and acute coronary syndrome unrelated to study treatment (n=1). The median age of patients was 62 years (range, 49-81 years), 79% of whom were men. BMS-986012 in combination with platinum/etoposide was well tolerated, and most treatment-related adverse events (TRAEs) were grade 1-2. The most common TRAEs (all grade; grade≥3) was pruritus (86%; 7%). In most cases, pruritus resolved with antihistamines or low dose corticosteroids. Urticaria (7%; 7%), neutropenia (7%; 7%), xerosis (7%; 0%), conjunctivitis (7%; 0%), infusion-related reaction (7%; 0%), and dizziness (7%; 0%) were also observed. No serious TRAEs or dose-limiting toxicities were reported. No notable differences were observed in the safety profiles of BMS-986012 plus cisplatin/etoposide (n=7) and BMS-986012 plus carboplatin/etoposide (n=7) in this study, and was comparable to the profile observed historically with platinum/etoposide chemotherapy alone, except for clinically manageable pruritus.

### Example 4

### Treatment of Human Subjects with Anti-Fucosyl-GM1 and Nivolumab

Human subjects were treated with a combination of anti-fucosyl-GM1 mAb BMS-986012 and anti-PD-1 mAb nivolumab in a Phase 1/2 Study (NCT02247349) to assess safety and efficacy, essentially as follows. Patients with relapsed/refractory small cell lung cancer (SCLC) who had not received prior checkpoint inhibitor (CPI) therapy (n=29) were treated Q3W intravenously with 400 mg (n=21) or 1000 mg (n=8) BMS-986012 and 360 mg nivolumab, with a median duration of follow-up of 18.6 months (range, 0.6-41.1 months). Median age of patients was 65 years (range, 46-79 years) and 52% were male; ECOG performance status was 0 (38%) or 1 (62%). All patients were current (24%) or former (76%) smokers. More patients were platinum sensitive (65%) than platinum refractory (30%).

The combination was well tolerated, with manageable adverse effects. The most common all-grade/grade≥3 treatment-related adverse events were pruritus (93%/21%), fatigue (28%/0%), dry skin (28%/0%), and hypothyroidism (17%/0%). In most patients, pruritus diminished over time and was managed with antihistamines and low-dose corticosteroids. Confirmed ORR with BMS-986012 plus nivolumab was 38% (CR, n=1 [3%]; PR, n=10 [35%]), with 3 additional patients (10%) having SD, for an overall disease control rate of 48%. This compares with a published ORR of 12% and a disease control rate of 29% for nivolumab monotherapy. Ready et al. (2020) J. Thorac. Oncol. 15:426 (147 patients).

At data cutoff, mDOR was 26.4 months (95% CI, 4.4 months—NR); 4 patients were still on therapy. mPFS was 2.1 months (95% CI, 1.4-9.9 months) and mOS was 18.7 months (95% CI, 8.2 months—NR). No differences in response were noted between platinum-sensitive and refractory populations.

The results presented here suggest that BMS-986012 plus nivolumab is a promising therapeutic combination for the treatment of patients with relapsed/refractory SCLC not previously treated with CPI therapy, irrespective of whether their cancers were platinum sensitive or refractory. The safety profile was manageable, and although based on a small number of patients, responses were clinically meaningful and durable.

### Example 5

### Combination Therapy with Carboplatin, Etoposide, Anti-Fucosyl-GM1 and Nivolumab

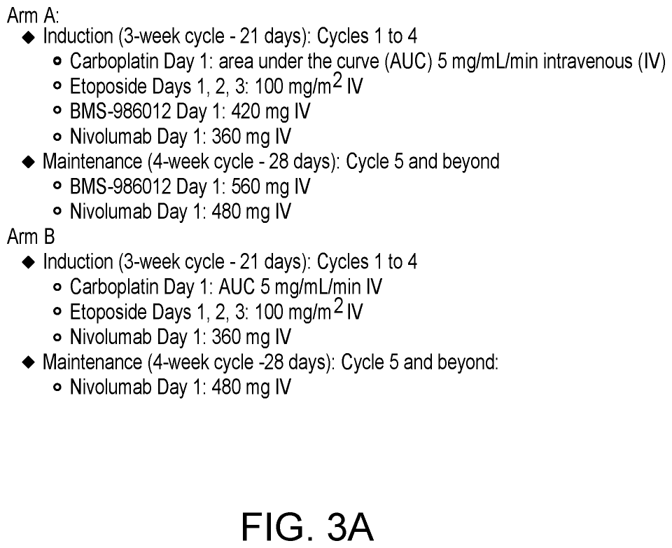

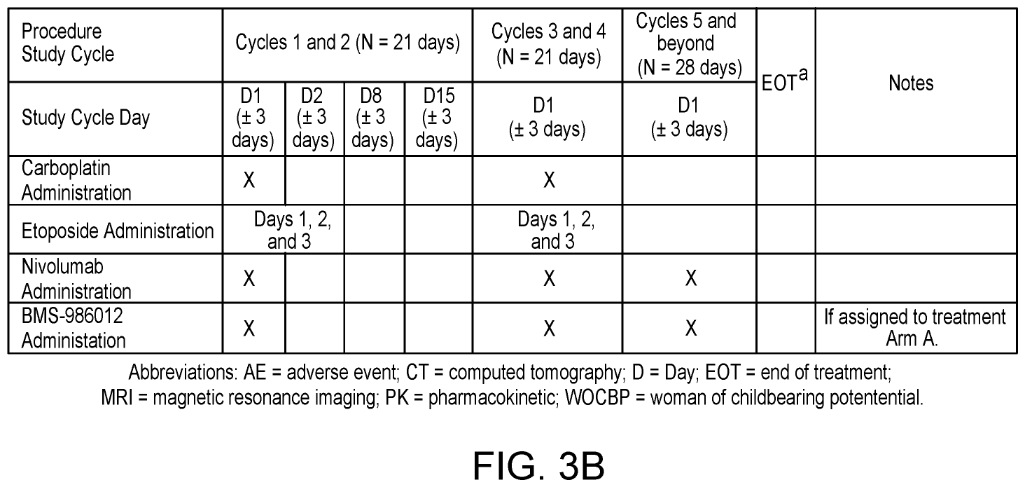

Patients afflicted with SCLC or ES-SCLC may be treated essentially as follows, and as illustrated at FIG. 3A. Patients are treated for four 21-day rounds of induction therapy comprising administration of carboplatin at AUC 5 mg/ml/min, etoposide at 100 mg/m², anti-fucosyl-GM1 mAb BMS-986012 at 420 mg, and anti-PD-1 mAb nivolumab at 360 mg, all iv on day one of each cycle. Etoposide is also administered at the same dose on days 2 and 3 of each cycle.

After four cycles of induction therapy, patients are treated with one or more 28-day rounds of maintenance therapy comprising administration of BMS-986012 at 560 mg, and anti-PD-1 mAb nivolumab at 480 mg, all iv on day one of each cycle.

TABLE 1
SUMMARY OF SEQUENCE LISTING

| SEQ ID NO. | Description |
|---|---|
| 1 | Anti-Fucosyl-GM1 Heavy Chain Variable Domain |
| 2 | Anti-Fucosyl-GM1 Light Chain Variable Domain |
| 3 | Anti-Fucosyl-GM1 Heavy Chain |
| 4 | Anti-Fucosyl-GM1 Light Chain |
| 5 | Anti-Fucosyl-GM1 CDRH1 |
| 6 | Anti-Fucosyl-GM1 CDRH2 |
| 7 | Anti-Fucosyl-GM1 CDRH3 |
| 8 | Anti-Fucosyl-GM1 CDRL1 |
| 9 | Anti-Fucosyl-GM1 CDRL2 |
| 10 | Anti-Fucosyl-GM1 CDRL3 |
| 11 | Anti-PD-1 Heavy Chain Variable Domain |
| 12 | Anti-PD-1 Light Chain Variable Domain |
| 13 | Anti-PD-1 Heavy Chain |
| 14 | Anti-PD-1 Light Chain |
| 15 | Anti-PD-1 CDRH1 |
| 16 | Anti-PD-1 CDRH2 |
| 17 | Anti-PD-1 CDRH3 |
| 18 | Anti-PD-1 CDRL1 |
| 19 | Anti-PD-1 CDRL2 |
| 20 | Anti-PD-1 CDRL3 |
| 21 | huPD-1 - NP_005009 |

Antibody sequences in the Sequence Listing include the sequences of the mature variable regions of the heavy and light chains, i.e. the sequences do not include signal peptides.

Equivalents: Those skilled in the art will recognize, or be able to ascertain using no more than routine experimentation, many equivalents of the specific embodiments disclosed herein. Such equivalents are intended to be encompassed by the following claims.

1. A method for treating a subject afflicted with small cell lung cancer (SCLC), comprising one or more rounds of induction therapy and optionally one or more rounds of maintenance therapy, wherein:
a. each round of induction therapy comprises treatment, on day one, with carboplatin, etoposide, anti-fucosyl-GM1 antibody and anti-PD-1 or anti-PD-L1 antibody; and
b. each round of maintenance therapy comprises treatment, on day one, with anti-fucosyl-GM1 antibody and anti-PD-1 antibody.

2. The method of claim 1, wherein the subject is afflicted with extensive-stage small cell lung cancer (ES-SCLC).

3. The method of claim 1 wherein each round of induction therapy is 21 days long (Q3W) and each round of maintenance therapy is 28 days long (Q4W).

4. The method of claim 1, wherein the anti-fucosyl-GM1 antibody cross-competes with BMS-986012 for binding to fucosyl-GM1, and further wherein BMS-986012 comprises heavy and light chain variable regions comprising the sequences of SEQ ID NOs: 1 and 2, respectively.

5. The method of claim 4, wherein the anti-fucosyl-GM1 antibody comprises:
a. a heavy chain comprising:
i. CDRH1 comprising the sequence of SEQ ID NO: 5;
ii. CDRH2 comprising the sequence of SEQ ID NO: 6; and
iii. CDRH3 comprising the sequence of SEQ ID NO: 7;
and
b. a light chain comprising:
i. CDRL1 comprising the sequence of SEQ ID NO: 8;
ii. CDRL2 comprising the sequence of SEQ ID NO: 9; and
iii. CDRL3 comprising the sequence of SEQ ID NO: 10.

6. The method of claim 5, wherein the anti-fucosyl-GM1 antibody comprises:
a. a heavy chain variable region comprising the sequence of SEQ ID NO: 1; and
b. a light chain variable region comprising the sequence of SEQ ID NO: 2.

7. The method of claim 6, wherein the anti-fucosyl-GM1 antibody comprises:
a. a heavy chain comprising the sequence of SEQ ID NO: 3; and
b. a light chain comprising the sequence of SEQ ID NO: 4; and wherein the anti-fucosyl-GM1 antibody is non-fucosylated.

8. (canceled)

9. The method of claim 7, wherein the anti-PD-1 or anti-PD-L1 antibody is an anti-PD-1 antibody comprising:
a. a heavy chain comprising the sequence of SEQ ID NO: 13; and
b. a light chain comprising the sequence of SEQ ID NO: 14.

10-12. (canceled)

13. The method of claim 9, wherein the anti-fucosyl-GM1 antibody is administered at 420 mg for induction therapy.

14. The method of claim 9, wherein the anti-fucosyl-GM1 antibody is administered at 560 mg for maintenance therapy.

15. The method of claim 9, wherein the anti-PD-1 antibody is administered at 360 mg for induction therapy.

16. The method of claim 9, wherein the anti-PD-1 antibody is administered at 480 mg for maintenance therapy.

17-19. (canceled)

20. The method of claim 1 comprising exactly four rounds of induction therapy.

21. The method of claim 20 comprising:
a. four 21-day rounds of induction therapy comprising iv administration, on day one of each round, of:
i) carboplatin AUC 5 mg/ml/min;
ii) etoposide at 100 mg/m²;
iii) BMS-986012 at 420 mg; and
iv) nivolumab at 360 mg; and
b. administration of etoposide at 100 mg/m² on days two and three of each of the four 21-day rounds of induction therapy; and
c. one or more 28-day rounds of maintenance therapy comprising iv administration, on day one of each round of maintenance therapy, of:
i) BMS-986012 at 560 mg; and
ii) nivolumab at 480 mg.

22. A method for treating a subject afflicted with small cell lung cancer (SCLC), comprising administering to the subject a therapeutically effective combination of:
a. an anti-fucosyl-GM1 antibody administered at a dose of 400 mg or 1000 mg, and;
b. an immunomodulatory agent selected from the group consisting of:
i. an anti-PD-1 antibody administered at a dose of 360 mg or 480 mg; and
ii. an anti-PD-L1 antibody administered at a dose of 1200 mg.

23. The method of claim 22, wherein the subject is afflicted with extensive-stage small cell lung cancer (ES-SCLC).

24-27. (canceled)

28. The method of claim 22, wherein the anti-fucosyl-GM1 antibody comprises:
a. a heavy chain comprising the sequence of SEQ ID NO: 3; and
b. a light chain comprising the sequence of SEQ ID NO: 4; and wherein the anti-fucosyl-GM1 antibody is non-fucosylated.

29-30. (canceled)

31. The method of claim 28, wherein the immunomodulatory agent is an anti-PD-1 antibody comprising:
a. a heavy chain comprising the sequence of SEQ ID NO: 13; and
b. a light chain comprising the sequence of SEQ ID NO: 14.

32-37. (canceled)

38. The method of claim 31, wherein the anti-fucosyl-GM1 antibody and the anti-PD-1 antibody are both administered on the same day Q3W or Q4W.

39. (canceled)

40. The method of claim 38, wherein the anti-fucosyl-GM1 antibody is BMS-986012 and the anti-PD-1 antibody is nivolumab, and further wherein BMS-986012 is administered at 400 or 1000 mg, and nivolumab is administered at 480 mg, and both are administered on the same day Q4W.


SEQUENCE LISTING (21 sequences)

SEQ ID NO: 1 - protein, 122 aa, Homo sapiens
EVQLVESGGGSVQPGESLRLSCVASGFTFSRYKMNWVRQAPGKGLEWVSYISRSGRDIYYADSVKGRFTISRDNAKNSLYLQMNSLRDEDTAVYYCAGTVTTYYYDFGMDVWGQGTTVTVSS

SEQ ID NO: 2 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIK

SEQ ID NO: 3 - protein, 451 aa, Homo sapiens
EVQLVESGGGSVQPGESLRLSCVASGFTFSRYKMNWVRQAPGKGLEWVSYISRSGRDIYYADSVKGRFTISRDNAKNSLYLQMNSLRDEDTAVYYCAGTVTTYYYDFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 214 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 5 aa, Homo sapiens
RYKMN

SEQ ID NO: 6 - protein, 17 aa, Homo sapiens
YISRSGRDIYYADSVKG

SEQ ID NO: 7 - protein, 13 aa, Homo sapiens
TVTTYYYDFGMDV

SEQ ID NO: 8 - protein, 11 aa, Homo sapiens
RASQGISSWLA

SEQ ID NO: 9 - protein, 7 aa, Homo sapiens
AASSLQS

SEQ ID NO: 10 - protein, 9 aa, Homo sapiens
QQYNSYPPT

SEQ ID NO: 11 - protein, 113 aa, Homo sapiens [sequence omitted]

SEQ ID NO: 12 - protein, 107 aa, Homo sapiens [sequence omitted]

SEQ ID NO: 13 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 5 aa, Homo sapiens
NSGMH

SEQ ID NO: 16 - protein, 17 aa, Homo sapiens
VIWYDGSKRYYADSVKG

SEQ ID NO: 17 - protein, 4 aa, Homo sapiens
NDDY

SEQ ID NO: 18 - protein, 11 aa, Homo sapiens
RASQSVSSYLA

SEQ ID NO: 19 - protein, 7 aa, Homo sapiens
DASNRAT

SEQ ID NO: 20 - protein, 9 aa, Homo sapiens
QQSSNWPRT

SEQ ID NO: 21 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL